(19) United States
(12) Patent Application Publication
(10) Pub. No.: US 2020/0088704 A1
(43) Pub. Date: Mar. 19, 2020

(54) FLOW CELL AND GAS ANALYZING DEVICE HAVING THE SAME

(71) Applicant: Shimadzu Corporation, Kyoto-shi (JP)

(72) Inventor: Fumiaki Otera, Kyoto-shi (JP)

(73) Assignee: Shimadzu Corporation, Kyoto-shi (JP)

(21) Appl. No.: 16/131,797

(22) Filed: Sep. 14, 2018

(51) Int. Cl. G01N 33/00; G01N 21/39

(52) U.S. Cl. CPC G01N 33/0031; H01M 8/18; G01N 2021/399; G01N 21/39

(57) ABSTRACT

A flow cell has a tubular main body and a thermocouple. An internal space is formed in the main body, a plurality of opening portions allowing the internal space to communicate with an external portion is formed in a peripheral wall, and sample gas flowing from at least one of the plurality of opening portions passes through the internal space and flows out of the other opening portion. The thermocouple includes two metal wires which are integrally attached to the main body, and a joint part of the two metal wires is provided in the vicinity (proximate) of the opening portion closer to an upstream side of the sample gas in a distributing direction than a center axis line of the main body.

### CROSS REFERENCE TO RELATED APPLICATIONS

This application relates to, but does not claim priority from, JP Ser. No.: JP2016-052493 filed Mar. 16, 2016 and published as JP Pub. No.: JP 2017-166978 on Sep. 21, 2017, the entire contents of which are incorporated herein fully by reference.

### BACKGROUND OF THE INVENTION

### Field of the Invention

The present invention relates to a flow cell in which sample gas passes through an internal space formed within a tubular main body, and a gas analyzing device having the flow cell.

### Description of the Related Art

As one of methods for measuring a concentration or a partial pressure of a component to be measured in sample gas, an absorption spectroscopy has been known. In the absorption spectroscopy, the sample gas is supplied into the measuring cell and light is irradiated into the measuring cell, so that the concentration or the partial pressure of the component to be measured is measured on the basis of spectrum obtained by a spectral operation of the transmitted light from the inside of the measuring cell.

Among methods for the absorption spectroscopy, a method using a semiconductor laser light source which can change an oscillation wavelength is called Tunable Diode Laser Absorption Spectroscopy (TDLAS). In the TDLAS, since the light source itself is a monochromatic light, any spectroscope is not required, and it is possible to simplify a device structure. Further, since the laser light having a high straightness is used in the TDLAS, it is possible to dispose a measuring cell having a long light path in a narrow space.

In the gas analyzing device which carries out an analysis by using the TDLAS, for example, a temperature sensor and a pressure sensor are provided within the measuring cell (refer, for example, to JP-A-2013-50403). When measuring the concentration or the partial pressure of the component to be measured in the sample gas, the temperature within the measuring cell is detected by the temperature sensor and the pressure within the measuring cell is detected by the pressure sensor, so that an arithmetic operation is carried out on the basis of these detected values.

### ASPECTS AND SUMMARY OF THE INVENTION

Of the temperature sensor and the pressure sensor provided in the gas analyzing device as mentioned above, the pressure sensor has comparatively small dispersion of a detected value in correspondence to its installation position. On the contrary, the temperature sensor has comparatively large dispersion of the detected value in correspondence to its installation position since the detected value tends to be affected by a temperature distribution. Therefore, in order for more precise measurement, the installation position of the temperature sensor is important.

Further, since the absorption spectroscopy using the laser light is characterized by a quick response, a responsiveness of the temperature sensor is also important. In general, in the case that a high-speed responsiveness is demanded, a thermocouple having a comparatively small heat capacity is used as the temperature sensor. However, in the case that an extremely high-speed responsiveness, for example, about 10 msec of time constant is demanded, it is necessary to prepare an extra-fine thermocouple. Therefore, there is a problem that it is hard to handle the thermocouple in terms of strength.

The present invention is made by taking the above actual condition into consideration, and an object of the present invention is to provide a flow cell which can suppress strength reduction and can carry out a measurement with a high responsiveness and more precisely, and a gas analyzing device having the flow cell.

A flow cell according to the present invention is provided with a tubular main body and a thermocouple. An internal space is formed in the main body, a plurality of opening portions allowing the internal space to communicate with an external portion is formed in a peripheral wall, and sample gas flowing from at least one of the plurality of opening portions passes through the internal space and flows out of the other opening portion. The thermocouple includes two metal wires which are integrally attached to the main body, and a joint part of the two metal wires is provided in the vicinity (proximate) of the opening portion closer to an upstream side of the sample gas in a distributing direction than a center axis line of the main body.

According to the structure mentioned above, since the thermocouple is integrally attached to the main body, it is possible to suppress the strength reduction even in the case of using an extra-fine thermocouple. Further, the responsiveness can be improved by using an extra-fine thermocouple having a small heat capacity.

Further, since the joint part of two metal wires constructing the thermocouple is provided in the vicinity (proximate) of the opening portion closer to the upstream side of the sample gas in the distributing direction than the center axis line of the main body, the joint part can be brought into direct contact with the sample gas flowing into the internal space from the opening portion, immediately before the inflow or immediately after the inflow. As a result, it is possible to directly detect the temperature of the sample gas before the heat of the sample gas is drawn by the main body of the flow cell, and it is accordingly possible to more precisely carry out a measurement.

The other flow cell according to the present invention is provided with a tubular main body and a plurality of thermocouples. An internal space is formed in the main body, a plurality of opening portions allowing the internal space to communicate with an external portion is formed in a peripheral wall, and the sample gas flowing from at least one of the plurality of opening portions passes through the internal space and flows out of the other opening portion. Each of the plurality of thermocouples includes two metal wires which are integrally attached to the main body respectively. Joint parts of the two metal wires are provided in the vicinity (proximate) of the plurality of opening portions at different angular positions in relation to the center axis line of the main body.

According to the structure mentioned above, since the plurality of thermocouples is integrally attached to the main body, it is possible to suppress strength reduction even in the case that extra-fine thermocouples are used. Further, the responsiveness can be improved by using the extra-fine thermocouples having the small heat capacity.

Further, since the joint parts of two metal wires constructing each of the thermocouples are provided in the vicinity (proximate) of the plurality of opening portions at the different angular positions in relation to the center axis line of the main body, the joint part can be brought into direct contact with the sample gas immediately before the inflow or immediately after the inflow, no matter from what opening portion the sample gas flows into the internal space. As a result, it is possible to directly detect the temperature of the sample gas before the heat of the sample gas is drawn by the main body of the flow cell regardless of the installation angle of the flow cell or the distributing direction of the sample gas, and it is accordingly possible to more precisely carry out the measurement.

A gas analyzing device according to the present invention is provided with the flow cell, a light source portion, a light receiving portion and a control portion. The light source portion irradiates light to the internal space of the flow cell. The light receiving portion receives the light that has passed through the internal space of the flow cell. The control portion carries out an arithmetic operation on the basis of a light receiving intensity of the light receiving portion and a thermal electromotive force of the thermocouple.

According to the present invention, it is possible to suppress the strength reduction even in the case that the extra-fine thermocouple is used. Further, the responsiveness can be improved by using the extra-fine thermocouple having the small heat capacity. Further, it is possible to directly detect the temperature of the sample gas before the heat of the sample gas is drawn by the main body of the flow cell, and it is accordingly possible to more precisely carry out the measurement.

The above and other aspects, features and advantages of the present invention will become apparent from the following description read in conjunction with the accompanying drawings, in which like reference numerals designate the same elements.

### BRIEF DESCRIPTION OF THE DRAWINGS

FIG. 1 is a schematic side elevational view showing an exemplary construction of a gas analyzing device according to a first embodiment of the present invention.

FIG. 2A is a schematic perspective view showing a specific construction in the vicinity (proximate) of an opening portion in a main body of a flow cell in FIG. 1.

FIG. 2B is a schematic cross sectional view showing the specific construction in the vicinity (proximate) of the opening portion in the main body of the flow cell in FIG. 1.

FIG. 3 is a view showing an example of a time-dependent change of a temperature of sample gas which is detected by using a thermocouple.

FIG. 4 is a schematic side elevation view showing an exemplary construction of a gas analyzing device according to a second embodiment of the present invention.

FIG. 5A is a schematic perspective view showing a specific construction in the vicinity (proximate) of an opening portion in a main body of a flow cell in FIG. 4.

FIG. 5B is a schematic cross sectional view showing the specific construction in the vicinity (proximate) of the opening portion in the main body of the flow cell in FIG. 4.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

### First Embodiment

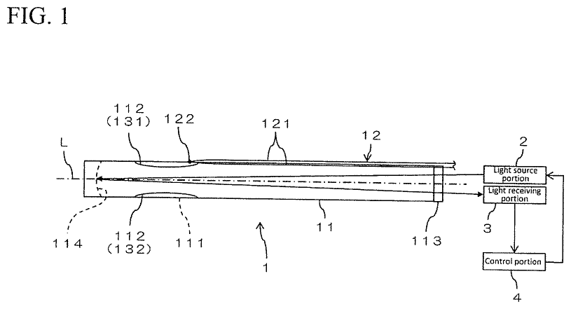

FIG. 1 is a schematic side elevational view showing an exemplary construction of a gas analyzing device according to a first embodiment of the present invention. The gas analyzing device according to the present embodiment is provided with a flow cell 1, a light source portion 2, a light receiving portion 3 and a control portion 4, and can measure concentration or partial pressure of a component to be measured in the sample gas, for example, using the TDLAS. The gas analyzing device can be applied, for example, to measurement of intake gas or exhaust gas of a motor vehicle, gas in a gas flue, gas generated in a plant facility, and gas in vacuum.

The flow cell 1 is provided with a tubular main body 11, and one thermocouple 12. In this example, the main body 11 is formed into a circular tube shape. The thermocouple 12 is integrally attached to the main body 11 by being directly fixed to a peripheral wall of the circular tubular main body 11.

The main body 11 extends in a straight line along a center axis line L. An internal space 111 extending in a straight line along the center axis line L is formed by forming an interior of the main body 11 into a hollow shape. A plurality of opening portions 112 is formed in the peripheral wall of the main body 11, and the internal space 111 is communicated with an external portion of the main body 11 via these opening portions 112.

The thermocouple 12 includes two metal wires 121. Two metal wires 121 are respectively extra-fine wire rods which are formed by different metal materials, and have diameters, for example, about 10 to 50 μm. Two metal wires 121 extend approximately in parallel to each other along the center axis line L, and a joint part 122 is formed by joining respective leading ends.

The joint part 122 of two metal wires 121 is provided in the vicinity (proximate) of one opening portion 112 among a plurality of opening portions 112. In this example, the joint part 122 is provided at a position which is opposite to the one opening portion 112 from an outer side of the main body 11.

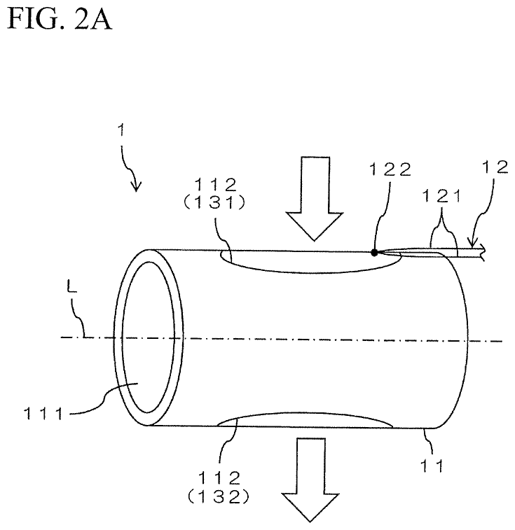

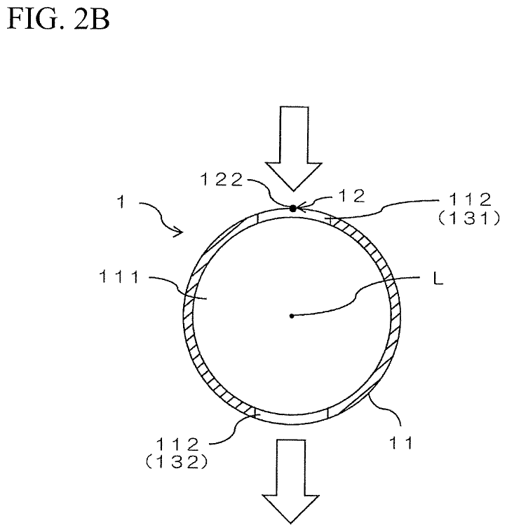

FIGS. 2A and 2B are views showing a specific construction of the vicinity (proximate) of the opening portion 112 in the main body 11 of the flow cell 1 in FIG. 1, and FIG. 2A shows a schematic perspective view and FIG. 2B shows a schematic cross sectional view, respectively.

In the present embodiment, two opening portions 112 are formed in the peripheral wall of the main body 11. Two opening portions 112 are formed so as to be spaced from each other in a peripheral direction, for example, by being formed at different angular positions in relation to the center axis line L. In this example, two opening portions 112 are formed at even intervals in the peripheral direction by being formed at the angular positions which are 180 degrees different from each other in relation to the center axis line L. As a result, two opening portions 112 are opposed to each other in relation to the center axis line L.

As shown by outline arrows in FIGS. 2A and 2B, the sample gas flows into the internal space 111 from one opening portion 112, and flows out of the other opening portion 112 through the internal space 111. More specifically, the one opening portion 112 constructs an inflow port 131 of the sample gas in relation to the internal space 111, and the other opening portion 112 constructs an outflow port 132 of the sample gas in relation to the internal space 111.

The thermocouple 12 is structured such as to detect the temperature of the sample gas passing through the internal space 111 as mentioned above, and the joint part 122 is provided in the vicinity (proximate) of the inflow port 131. As a result, the joint part 122 of the thermocouple 12 is provided closer to an upstream side of the sample gas in a distributing direction than the center axis line L of the main body 11, and faces directly to the flow of the sample gas.

With reference again to FIG. 1, a transparent window 113 is provided in one end surface of the main body 11. The light source portion 2 and the light receiving portion 3 are arranged so as to be opposed to the window 113 along the center axis line L. A reflecting mirror 114 is provided in the other end surface of the main body 11 in a posture where a reflective surface is directed to the internal space 111 side. The reflective surface of the reflecting mirror 114 is constructed, for example, by a spherical concave surface.

The light source portion 2 is constructed, for example, by a semiconductor laser diode. The light (laser light) irradiated from the light source portion 2 enters into the internal space 111 of the flow cell 1 from the window 113. The light entering into the internal space 111 is directed from one end side to the other end side in the internal space 111 along the center axis line L of the main body 11, is reflected by the reflective surface of the reflecting mirror 114 and is thereafter returned again to the one end side so as to be emitted to the external portion of the main body 11 from the window 113. The light passing through the internal space 111 and emitted from the window 113 as mentioned above is received in the light receiving portion 3, for example, constructed by a photodiode.

The light passing through the internal space 111 of the main body 11 transmits the sample gas which flows into the internal space 111 from the inflow port 131. In this example, the distributing direction of the sample gas in relation to the internal space 111 is orthogonal to a direction that the light passes through the internal space 111 (a direction that the center axis line L extends). When the light transmits the sample gas, the light of a specific wavelength is absorbed depending on a component to be treated as a measuring object in the sample gas (a component to be measured). As a result, the light receiving intensity (the transmitted light intensity) of each of the wavelengths in the light receiving portion 3 varies depending on the component to be measured.

The control portion 4 is structured, for example, such as to include a central processing unit (CPU), and carries out an arithmetic operation on the basis of the input signal from the light receiving portion 3 as well as controlling a motion of the light source portion 2. Since the control portion 4 carries out a current control and a temperature control in relation to the light source portion 2, the light (for example, the infrared light) of the specific wavelength band is irradiated from the light source portion 2.

The arithmetic operation carried out by the control portion 4 is carried out on the basis of not only the light receiving intensity in the light receiving portion 3 but also the thermal electromotive force of the thermocouple 12 and the pressure within the flow cell 1 detected by the pressure sensor (not shown). A description will be specifically given below of an aspect of the arithmetic operation carried out by the control portion 4.

The light receiving intensity in the light receiving portion 3 has a relationship as shown by the following expression (1) according to the Lambert-Beer law. Here, ν denotes a frequency of the laser light, I₀(ν) denotes a light receiving intensity of the laser light in the case that the component to be measured is not absorbed in the frequency ν, I(ν) denotes a light receiving intensity of the laser light in the case that the component to be measured is absorbed in the frequency ν, c denotes a number density of molecules of the component to be measured, l denotes a light path length of the laser light passing through the sample gas, S(T) denotes an absorption line intensity of the sample gas at the gas temperature T, and K(ν) denotes an absorption property function.

$$\begin{matrix}[ Numerical Expression 1 ] & \\ ln ( \frac{{I}_{0} ( v )}{I ( v )} ) = c \times l \times S ( T ) \times K ( v ) & ( 1 )\end{matrix}$$

In the case that the sample gas is near the atmospheric pressure, the absorption property function K(ν) is expressed by the Lorentz function as shown by the following expression (2).

$$\begin{matrix}[ Numerical Expression 2 ] & \\ K ( v ) = \frac{{γ}_{L}}{π \{ {( v - {v}_{0} )}^{2} + {γ}_{L}^{2} \}} & ( 2 )\end{matrix}$$

ν₀ denotes a center frequency of the absorption spectrum. γ_{L} denotes a half value width of the absorption spectrum, or so-called Lorentz width, and can be approximated as the following expression (3). Here, p denotes the pressure of the sample gas, T denotes the temperature of the sample gas, and γ_{L0} denotes a half value width of the absorption under the condition of p₀ and T₀. On the basis of this expression (3), it is known that the absorption spectrum depends on the temperature and the pressure of the sample gas. The temperature of the sample gas can be detected on the basis of the thermal electromotive force of the thermocouple 12. Further, the pressure of the sample gas is detected by the pressure sensor (not shown) which is provided within the flow cell 1.

[Numerical Expression 3]

γ_{L}=γ_{L0}(p/p₀)(T₀/T)^{1/2} (3)

The following expression (4) is established from the expression (1) and the expression (2) mentioned above. By using a laser extremely narrower than a line width of the absorption spectrum, for example, using a distributed feedback (DFB) type semiconductor laser, it is possible to carry out the measurement in the respective frequencies v without independently requiring any spectroscope.

$$\begin{matrix}[ Numerical Expression 4 ] & \\ ln ( \frac{{I}_{0} ( v )}{I ( v )} ) = c \times l \times S ( T ) \times \frac{{γ}_{L}}{π \{ {( v - {v}_{0} )}^{2} + {γ}_{L}^{2} \}} & ( 4 )\end{matrix}$$

On the basis of the expression (4) mentioned above, the light receiving intensity I(ν) of the laser light in the center frequency ν₀ satisfies the following expression (5). S(T) in the following expression (5) and the Lorentz width γ_{L0} in the normal state in the above expression (3) can be known about a lot of molecules by using a database called as HITRAN. Therefore, it is possible to determine the number density of molecules c of the component to be measured, by measuring I₀(ν₀) and I(ν₀).

$$\begin{matrix}[ Numerical Expression 5 ] & \\ ln ( \frac{{I}_{0} ( {v}_{0} )}{I ( {v}_{0} )} ) = c \times l \times S ( T ) \times \frac{1}{π {γ}_{L}} & ( 5 )\end{matrix}$$

In the present embodiment, since the thermocouple 12 is integrally attached to the main body 11 as described by using FIGS. 1, 2A and 2B, it is possible to suppress the strength reduction even in the case that the extra-fine thermocouple 12 is used. Further, in the case that the extra-fine thermocouple 12 having the small heat capacity is used, it is possible to improve the responsiveness.

Further, since the joint part 122 of two metal wires 121 constructing the thermocouple 12 is provided in the vicinity (proximate) of the opening portion 112 (the inflow port 131) closer to the upstream side of the sample gas in the distributing direction than the center axis line L of the main body 11, the joint part 122 can be brought into direct contact with the sample gas flowing into the internal space 111 from the opening portion 112 immediately before the inflow. As a result, it is possible to directly detect the temperature of the sample gas before the heat of the sample gas is drawn by the main body 11 of the flow cell 1, and it is accordingly possible to more precisely carry out the measurement.

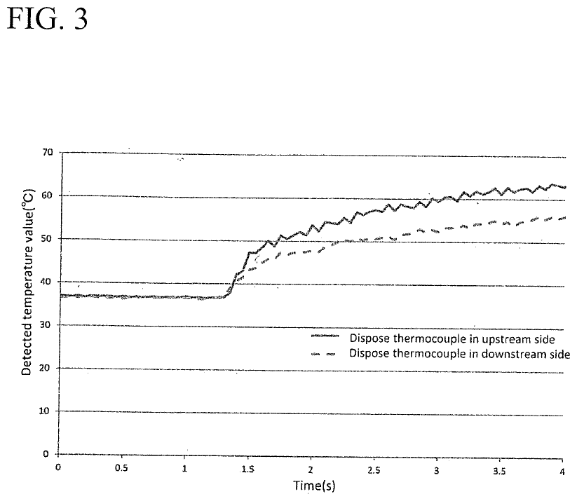

FIG. 3 is a view showing an example of a time-dependent change of the temperature of the sample gas which is detected by using the thermocouple 12. In this FIG. 3, the case that the joint part 122 of the thermocouple 12 is provided closer to the upstream side (the inflow port 131 side) of the sample gas in the distributing direction than the center axis line L of the main body 11 is shown by a solid line, and the case that the joint part 122 of the thermocouple 12 is provided closer to the downstream side (the outflow port 132 side) of the sample gas in the distributing direction than the center axis line L of the main body 11 is shown by a broken line.

On the basis of results of measurement shown in FIG. 3, it is known that when the sample gas having the high temperature flows into the internal space 111 from the inflow port 131, the detected temperature by the thermocouple 12 tends to rise in the case that the joint part 122 of the thermocouple 12 is provided in the inflow port 131 side (the solid line in FIG. 3) than in the case that the joint part 122 is provided in the outflow port 132 side (the broken line in FIG. 3). This means that the temperature of the sample gas can be detected before heat of the sample gas is drawn by the main body 11 of the flow cell 1 by the provision of the joint part 122 of the thermocouple 12 in the inflow port 131 side, and it is possible to more precisely carry out the measurement.

In the present embodiment, the description is given of the case that the number of the opening portion 112 formed in the main body 11 of the flow cell 1 is two constituted by the inflow port 131 and the outflow port 132. However, one or a plurality of opening portions 112 may be formed in the peripheral wall of the main body 11 in addition to the inflow port 131 and the outflow port 132, without being limited to the structure mentioned above. In this case, it is possible to employ such a structure that the sample gas flows into from a plurality of opening portions 112, or such a structure that the sample gas flows out of a plurality of opening portions 112.

A plurality of opening portions 112 may be structured such as to be formed at different intervals from each other in the peripheral direction, without being limited to be structured such as to be formed at even intervals each other in the peripheral direction. Further, a plurality of opening portions 112 may be structured, for example, not to be arranged side by side in the peripheral direction by being formed at deviated positions from each other along the direction that the center axis line L extends.

The joint part 122 of the thermocouple 12 is not limited to the structure in which the joint part 122 is provided at the opposite position to the inflow port 131 from the outer side of the main body 11, as long as the joint part 122 is provided closer to the upstream side of the sample gas in the distributing direction than the center axis line L of the main body 11. For example, the joint part 122 may be provided at a position which is opposed to the inflow port 131 from an inner side of the main body 11, and the joint part 122 may be accordingly brought into direct contact with the sample gas flowing into the internal space 111 from the inflow port 131 immediately after the inflow. Further, the joint part 122 may be provided at a position which is opposed to the peripheral edge portion of the opening portion 112 without being limited to the position which is opposed to the opening portion 112, as long as the position is near the opening portion 112.

In the present embodiment, the description is given of the case that the main body 11 of the flow cell 1 has the circular tube shape. However, the main body 11 of the flow cell 1 is not limited to the circular tube shape as long as the main body 11 is a hollow member in which the internal space 111 is formed, but can employ the other shapes such as an oval or rectangular cross section. Further, the flow cell 1 is not limited to the structure which is provided with the reflecting mirror 114, but may be structured, for example, such that the light irradiated into the internal space 111 from the light source portion 2 provided in one end side of the main body 11 passes through the internal space 111 and is received by the light receiving portion 3 provided in the other end side of the main body 11.

### Second Embodiment

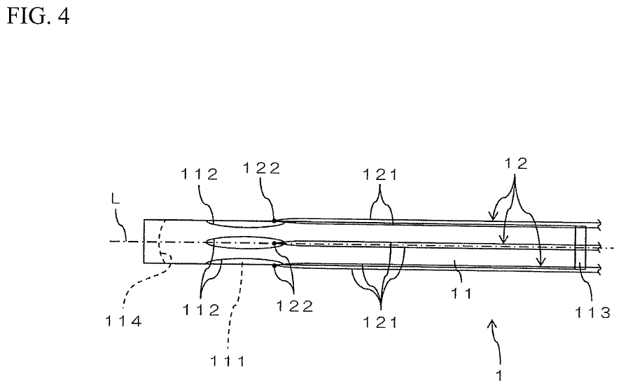

FIG. 4 is a schematic side elevational view showing an exemplary construction of a gas analyzing device according to a second embodiment of the present invention. The gas analyzing device according to the present embodiment is provided with a flow cell 1, a light source portion 2, a light receiving portion 3 and a control portion 4 in the same manner as the first embodiment, and can measure the concentration or the partial pressure of the component to be measured in the sample gas, for example, by using the TDLAS. Since the structures of the light source portion 2, the light receiving portion 3 and the control portion 4 are the same as those of the first embodiment, an illustration of these structures is omitted in FIG. 4.

The flow cell 1 is provided with a tubular main body 11, and a plurality of thermocouples 12. In this example, the main body 11 is formed into a circular tube shape. The plurality of thermocouples 12 is integrally attached to the main body 11 by being directly fixed to a peripheral wall of the circular tubular main body 11 respectively.

The main body 11 extends in a straight line along a center axis line L. An internal space 111 extending in a straight line along the center axis line L is formed by forming an interior of the main body 11 into a hollow shape. A plurality of opening portions 112 is formed in the peripheral wall of the main body 11, and the internal space 111 is communicated with an external portion of the main body 11 via these opening portions 112.

Each of the plurality of thermocouples 12 includes two metal wires 121. Two metal wires 121 are respectively extra-fine wire rods which are formed by different metal materials, and have diameters, for example, about 10 to 50 μm. Two metal wires 121 extend approximately in parallel to each other along the center axis line L, and a joint part 122 is formed by joining respective leading ends.

The joint parts 122 of two metal wires 121 in the respective thermocouples 12 are provided in the vicinity (proximate) of the opening portions 112 which are different from each other. In this example, the joint parts 122 of the thermocouples 12 which are different from each other are provided at positions which are opposite to the respective opening portions 112 from an outer side of the main body 11.

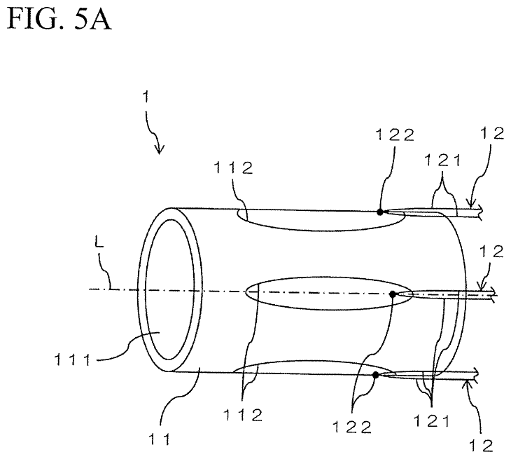

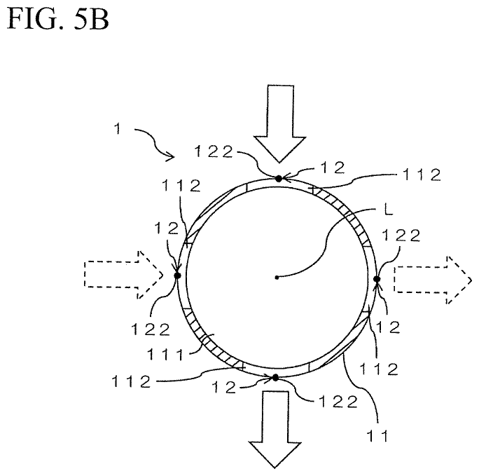

FIGS. 5A and 5B are views showing a specific construction of the vicinity (proximate) of the opening portion 112 in the main body 11 of the flow cell 1 in FIG. 4, and FIG. 5A shows a schematic perspective view and FIG. 5B shows a schematic cross sectional view, respectively.

In the present embodiment, four opening portions 112 are formed in the peripheral wall of the main body 11. Four opening portions 112 are formed so as to be spaced from each other in a peripheral direction, for example, by being formed at different angular positions in relation to the center axis line L. In this example, four opening portions 112 are formed at even intervals in the peripheral direction by being formed at the angular positions which are every 90 degrees different from each other in relation to the center axis line L. As a result, two of four opening portions 112 are opposed to each other in relation to the center axis line L, and the remaining two are opposed to each other in relation to the center axis line L.

In the flow cell 1 having the structure mentioned above, the sample gas can be flowed into the internal space 111 from any opening portion 112. For example, the sample gas mainly flows out of the opening portion 112 which is opposed to the opening portion 112 in relation to the center axis line L by flowing the sample gas into the internal space 111 from any one opening portion 112 as shown by an outline arrow of a solid line in FIG. 5B. Further, the sample gas mainly flows out of the opening portion 112 which is opposed to the opening portion 112 in relation to the center axis line L by flowing the sample gas into the internal space 111 from the other opening portion 112 as shown by an outline arrow of a broken line in FIG. 5B. More specifically, any opening portion 112 can be set to the inflow port or the outflow port among a plurality of opening portions 112.

Each of the thermocouples 12 is structured such as to detect the temperature of the sample gas passing through the internal space 111 as mentioned above, and the joint parts 122 of the respective thermocouples 12 are provided in the vicinity (proximate) of the plurality of opening portions 112, respectively. As a result, the joint parts 122 of the respective thermocouples 12 are provided at the different angular positions in relation to the center axis line L of the main body 11.

With reference again to FIG. 4, a transparent window 113 is provided in one end surface of the main body 11. Further, a reflecting mirror 114 is provided in the other end surface of the main body 11 in a posture where a reflective surface is directed to the internal space 111 side. The reflective surface of the reflecting mirror 114 is constructed, for example, by a spherical concave surface.

In the present embodiment, the light (laser light) irradiated from the light source portion 2 enters into the internal space 111 of the flow cell 1 from the window 113, and is directed from one end side to the other end side in the internal space 111 along the center axis line L of the main body 11, in the same manner as the first embodiment. Further, the light reflected by the reflective surface of the reflecting mirror 114 is again returned to the one end side so as to be emitted to the external portion of the main body 11 from the window 113, and is received in the light receiving portion 3.

The light passing through the internal space 111 of the main body 11 transmits the sample gas which flows into the internal space 111 from any opening portion 112. In this example, the distributing direction of the sample gas in relation to the internal space 111 is orthogonal to a direction that the light passes through the internal space 111 (a direction that the center axis line L extends). When the light transmits the sample gas, the light of a specific wavelength is absorbed depending on a component to be treated as a measuring object in the sample gas (a component to be measured). As a result, the light receiving intensity (the transmitted light intensity) of each of the wavelengths in the light receiving portion 3 varies depending on the component to be measured.

In the present embodiment, since the plurality of thermocouples 12 is integrally attached to the main body 11 as described by using FIGS. 4, 5A and 5B, it is possible to suppress the strength reduction even in the case that the extra-fine thermocouples 12 are used. Further, in the case that the extra-fine thermocouples 12 having the small heat capacity are used, it is possible to improve the responsiveness.

Further, since the joint part 122 of two metal wires 121 constructing each of the thermocouples 12 is provided in the vicinity (proximate) of the plurality of opening portions 112 at the different angular positions in relation to the center axis line L of the main body 11, the joint part 122 can be brought into direct contact with the sample gas immediately before the inflow, no matter from what opening portions 112 the sample gas flows into the internal space 111. As a result, it is possible to directly detect the temperature of the sample gas before the heat of the sample gas is drawn by the main body 11 of the flow cell 1 regardless of the installation angle of the flow cell 1 or the distributing direction of the sample gas, and it is accordingly possible to more precisely carry out the measurement.

The control portion 4 carries out an arithmetic operation by selecting an optimum temperature among the temperatures of the sample gas which are detected by the respective thermocouples 12. Specifically, in the case that the temperature of the sample gas rises, the highest temperature is selected among the temperatures of the sample gas which are detected by the respective thermocouples 12. On the contrary, in the case that the temperature of the sample gas lowers, the lowest temperature is selected among the temperatures of the sample gas which are detected by the respective thermocouples 12. As a result, even in the case that the installation angle of the flow cell 1 or the distributing direction of the sample gas is not known, the sample gas can be measured by using the detected value of the thermocouple 12 in which the joint part 122 is provided in the vicinity (proximate) of the opening portion 112 in the most upstream side of the sample gas in the distributing direction.

In the present embodiment, the description is given of the case that the number of the opening portion 112 formed in the main body 11 of the flow cell 1 is four. However, three or less or five or more opening portions 112 may be provided without being limited to four opening portions 112 as long as a plurality of opening portions 112 is provided at the different positions in relation to the center axis line L of the main body 11. In this case, it is possible to employ such a structure that the sample gas flows from a plurality of opening portions 112, or such a structure that the sample gas flows out of a plurality of opening portions 112.

A plurality of opening portions 112 may be structured such as to be formed at different intervals from each other in the peripheral direction, without being limited to be structured such as to be formed at even intervals each other in the peripheral direction. Further, a plurality of opening portions 112 may be structured, for example, not to be arranged side by side in the peripheral direction by being formed at deviated positions from each other along the direction that the center axis line L extends.

The joint part 122 of each of the thermocouples 12 is not limited to the structure in which the joint part 122 is provided at the opposite position to the opening portion 112 from the outer side of the main body 11, as long as the joint part 122 is provided in the vicinity (proximate) of the opening portion 112. For example, the joint part 122 may be provided at a position which is opposed to the opening portion 112 from an inner side of the main body 11, and the joint part 122 may be accordingly brought into direct contact with the sample gas flowing into the internal space 111 from the opening portion 112 immediately after the inflow. Further, the joint part 122 may be provided at a position which is opposed to the peripheral edge portion of the opening portion 112, without being limited to the position which is opposed to the opening portion 112.

In the present embodiment, the description is given of the case that the main body 11 of the flow cell 1 has the circular tube shape. However, the main body 11 of the flow cell 1 is not limited to the circular tube shape as long as the main body 11 is a hollow member in which the internal space 111 is formed, but can employ the other shapes such as an oval or rectangular cross section. Further, the flow cell 1 is not limited to the structure which is provided with the reflecting mirror 114, but may be structured, for example, such that the light irradiated into the internal space 111 from the light source portion 2 provided in one end side of the main body 11 passes through the internal space 111 and is received by the light receiving portion 3 provided in the other end side of the main body 11.

Although only a few embodiments have been disclosed in detail above, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art.

Also, the inventors intend that only those claims which use the words “means for” are intended to be interpreted under 35 USC 112 only when the word ‘means’ and ‘for’ are used together, next to each other in the form of “means for”, and not otherwise. Moreover, no limitations from the specification are intended to be read into any claims, unless those limitations are expressly included in the claims.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. A flow cell, comprising:
a tubular main body in which an internal space is formed in the main body, a plurality of opening portions allowing the internal space to communicate with an external portion are formed in a peripheral wall of the main body, and a sample gas flowing from at least one of the plurality of opening portions passes through the internal space and flows out of the other opening portion; and
a thermocouple which includes two metal wires integrally attached to the main body, and in which a joint part of the two metal wires is provided proximate the opening portion closer to an upstream side of the sample gas in a distributing direction than a center axis line of the main body.

2. A flow cell, comprising:
a tubular main body in which an internal space is formed in the main body, a plurality of opening portions allowing the internal space to communicate with an external portion is formed in a peripheral wall, and sample gas flowing from at least one of the plurality of opening portions passes through the internal space and flows out of the other opening portion; and
a plurality of thermocouples, each of which includes two metal wires integrally attached to the main body, and in which joint parts of the two metal wires are provided in the vicinity of the plurality of opening portions at different angular positions in relation to the center axis line of the main body.

3. A gas analyzing device, comprising:
the flow cell, according to claim 1;
a light source portion which irradiates light to the internal space of the flow cell;
a light receiving portion which receives the light that has passed through the internal space of the flow cell; and
a control portion which carries out an arithmetic operation on the basis of a light receiving intensity of the light receiving portion and a thermal electromotive force of the thermocouple.

4. A gas analyzing device, comprising:
the flow cell, according to claim 2;
a light source portion which irradiates light to the internal space of the flow cell;
a light receiving portion which receives the light that has passed through the internal space of the flow cell; and
a control portion which carries out an arithmetic operation on the basis of a light receiving intensity of the light receiving portion and a thermal electromotive force of the thermocouples.
